(19) United States
(12) Patent Application Publication
(10) Pub. No.: US 2021/0045778 A1
(43) Pub. Date: Feb. 18, 2021

(54) PEDICLE SCREW TULIP HEAD

(71) Applicant: Premia Spine Ltd., Ramat Poleg (IL)

(72) Inventors: Rafi Haziza, Kiryat Bialik (IL); Uri Arnin, Kiryat Tivon (IL)

(73) Assignee: Premia Spine Ltd., Ramat Poleg (IL)

(21) Appl. No.: 16/541,190

(22) Filed: Aug. 15, 2019

(51) Int. Cl. A61B 17/70

(52) U.S. Cl. CPC A61B 17/7032; A61B 17/7035

(57) ABSTRACT

A bone fastener includes a tulip head including sides spaced by an opening and a lower portion below the opening and extending from the sides. The lower portion is formed with a lower central opening configured to allow a portion of a pedicle screw to pass. The lower portion is formed with at least one aperture offset sideways from the lower central opening.

### FIELD OF THE INVENTION

The present invention is related generally to pedicle screws and particularly to a polyaxial tulip head for a pedicle screw with an increased range of polyaxial motion.

### BACKGROUND OF THE INVENTION

Many polyaxial pedicle screws have a tulip head. FIGS. 1A-1C illustrate a typical prior art pedicle screw 1 with a tulip head 2. The tulip head 2 has two opposing sides 3 spaced by an opening 4 to receive a spinal rod (not shown). The tulip head 2 is formed with internal threads 5 to receive a rod locking setscrew (not shown) to anchor or fix the rod in the tulip.

The pedicle screw 1 has a threaded shank 6 and a spherical head 7. A neck 8 is formed at the junction between the head 7 and the shank 6. The neck 8 is narrower than the portion of the shank 6 nearest the head 7. Often a longitudinal passageway 9 is formed through the head 7 and shank 6. The lower portion 11 of tulip head 2 is formed with a central opening 12 to allow the threaded shank 6 to pass. Often the tulip has a saddle 10, which typically has an upper recessed curvature in which the rod sits and a lower cup like opening to receive the top of the pedicle screw head 7. When the setscrew is tightened, it fixes the rod against the saddle at a desired angle.

The tulip head 2 can swivel in a polyaxial motion about the pedicle screw head 7. In this manner, the rod secured by the setscrew to the tulip head 2 can be fixed at many different rotational orientations with respect to the pedicle screw shank 6. The abutment of the lower portion 11 of the tulip head 2 against the pedicle screw determines the maximum angular range of polyaxial motion of the tulip head.

### SUMMARY

The present invention seeks to provide a polyaxial tulip head for a pedicle screw with an increased range of polyaxial motion, as is described more in detail hereinbelow.

There is thus provided in accordance with a non-limiting embodiment of the present invention a bone fastener including a tulip head including sides spaced by an opening and a lower portion below the opening and extending from the sides, the lower portion being formed with a lower central opening configured to allow a portion of a pedicle screw to pass, and wherein the lower portion is formed with at least one aperture offset sideways from the lower central opening.

In accordance with a non-limiting embodiment of the invention the at least one aperture is formed along a lower curvature of the lower portion.

In accordance with a non-limiting embodiment of the invention the at least one aperture is formed through an entire thickness of the lower portion.

In accordance with another non-limiting embodiment of the invention the at least one aperture is formed through a partial thickness of the lower portion.

In accordance with another non-limiting embodiment of the invention the at least one aperture includes more than one aperture at different circumferential positions around the lower portion. The apertures may have the same or different sizes and shapes. The tulip head may be formed with internal threads. The tulip head may include a saddle received in the lower portion.

The bone fastener further includes a pedicle screw that includes a threaded shank and a spherical head, wherein the head is received in the central opening so that the tulip head articulates about the head.

### BRIEF DESCRIPTION OF THE DRAWINGS

The present invention will be understood and appreciated more fully from the following detailed description taken in conjunction with the drawings in which:

FIG. 1A is a simplified side-view illustration of a prior art pedicle screw with a tulip head.

FIG. 1B is a simplified side-view illustration of the prior art pedicle screw with the tulip head inclined at its maximum angular range of polyaxial motion.

FIG. 1C is a simplified perspective illustration of the prior art tulip head.

FIG. 2A is a simplified side-view illustration of a pedicle screw with a tulip head, constructed and operative in accordance with a non-limiting embodiment of the invention.

FIG. 2B is a simplified side-view illustration of the pedicle screw with the tulip head of FIG. 2A inclined at its maximum angular range of polyaxial motion, which is greater than the maximum angular range of the prior art tulip head.

FIG. 2C is a simplified perspective illustration of the tulip head of FIGS. 2A-2B.

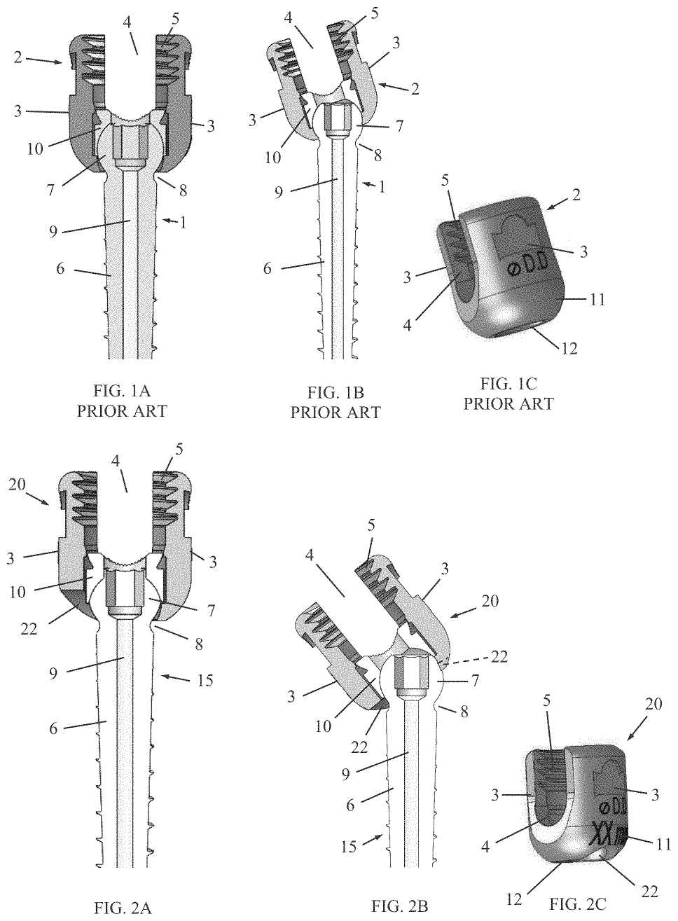

### DETAILED DESCRIPTION

Reference is now made to FIGS. 2A, 2B and 2C, which illustrate a pedicle screw 15 with a tulip head 20, constructed and operative in accordance with a non-limiting embodiment of the invention.

The pedicle screw 15 may be constructed as in the prior art, and as such, has a threaded shank 6 and a spherical head 7. A neck 8 is formed at the junction between the head 7 and the shank 6. The neck 8 is narrower than the portion of the shank 6 nearest the head 7. A longitudinal passageway 9 may be optionally formed through the head 7 and shank 6

Tulip head 20, similar to the prior art, may have two opposing sides 3 spaced by an opening 4 to receive a spinal rod (not shown). Tulip head 20 may be formed with internal threads 5 to receive a rod locking setscrew (not shown) to anchor or fix the rod in the tulip.

A lower portion 11 (which may be rounded) of tulip head 20 is below opening 4 and extends from sides 3. The lower portion 11 may be formed with a lower central opening 12 to allow a portion of the pedicle screw (e.g., the threaded shank 6) to pass. The head 7 is received in the central opening 12 so that tulip head 20 articulates about pedicle screw head 7. Tulip head 20 may have a saddle 10 received in lower portion 11. Saddle 10 may have an upper recessed curvature in which the rod sits and a lower cup like opening to receive the top of the pedicle screw head 7. (Throughout, upper and lower are in terms of the drawings, but not necessarily in terms of the particular installation of the pedicle screw and tulip head.) As in the prior art, when the setscrew is tightened, it fixes the rod against the saddle at a desired angle.

In accordance with a non-limiting embodiment of the invention, the lower portion 11 tulip head 20 is formed with at least one aperture 22 offset sideways (radially offset) from the lower central opening 12. As seen in FIG. 2B, due to the aperture 22, the inclination of the tulip head 20 with respect to the shank 6 of pedicle screw 15 is greater than the inclination of the prior art tulip head 2 with respect to the shank 6 of the prior art pedicle screw 1. Due to the aperture 22, the tulip head 20 abuts against either the head 7 or neck 8 of the pedicle screw 15 at a greater inclination than the prior art tulip head 2.

The aperture 22 may be formed along the lower curvature of the lower portion 11. The aperture 22 may be formed through the entire thickness of the lower portion 11 or alternatively may be blind, that is, not formed through the entire thickness of the lower portion 11 but only through a partial thickness of the lower portion 11.

In one embodiment, only one aperture 22 is present. However, as seen in broken lines in FIG. 2B, more than one aperture 22 may be formed in tulip head 20, such as opposing apertures 22 or at different circumferential positions around the lower portion 11 of the tulip head 20. When more than one aperture 22 is used, the apertures may be of the same size and shape or may be different sizes and shapes (e.g., elliptical or oblong as opposed to circular).

What is claimed is:

1. A bone fastener comprising:
a tulip head comprising sides spaced by an opening and a lower portion below said opening and extending from said sides, said lower portion being formed with a lower central opening configured to allow a portion of a pedicle screw to pass, and wherein said lower portion is formed with at least one aperture offset sideways from said lower central opening.

2. The bone fastener according to claim 1, wherein said at least one aperture is formed along a lower curvature of said lower portion.

3. The bone fastener according to claim 1, wherein said at least one aperture is formed through an entire thickness of said lower portion.

4. The bone fastener according to claim 1, wherein said at least one aperture is formed through a partial thickness of said lower portion.

5. The bone fastener according to claim 1, wherein said at least one aperture comprises more than one aperture at different circumferential positions around said lower portion.

6. The bone fastener according to claim 5, wherein said apertures have same sizes and shapes.

7. The bone fastener according to claim 5, wherein said apertures have different sizes or shapes.

8. The bone fastener according to claim 1, wherein said tulip head is formed with internal threads.

9. The bone fastener according to claim 1, wherein said tulip head comprises a saddle received in said lower portion.

10. The bone fastener according to claim 1, further comprising a pedicle screw that comprises a threaded shank and a spherical head, wherein said head is received in said central opening so that said tulip head articulates about said head.
